(19) United States
(12) Patent Application Publication
(10) Pub. No.: US 2026/0315668 A1
(43) Pub. Date: Oct. 15, 2026

(54) WEARABLE BREAST PUMP HAVING FUNCTION OF VIBRATION AND MASSAGE

(71) Applicant: Shenzhen TPH Technology Co., Ltd., Shenzhen, Guangdong (CN)

(72) Inventor: Xianzi CHEN, Shenzhen, Guangdong (CN)

(21) Appl. No.: 19/148,793

(22) PCT Filed: Mar. 31, 2023
(86) PCT No.: PCT/CN2023/085688

§ 371 Date: Jul. 17, 2025

(30) Foreign Application Priority Data
Nov. 17, 2022 (CN) 202211443191.X

(51) Int. Cl. A61M 1/06

(52) U.S. Cl. CPC A61M 1/0697; A61M 1/064; A61M 1/067; A61M 1/0693; A61M 2205/502; A61M 2205/8206; A61M 2205/8237

(57) ABSTRACT

The wearable breast pump having a function of vibration and massage claimed in the present invention comprises a breast pump cup, a massage portion and a vibrating massaging unit, wherein the massage portion is disposed on the breast pump cup, the vibrating massaging unit is correspondingly disposed on the massage portion, and the vibrating massaging unit produces a vibration under the action of electric current. In the present invention, by way of arranging the vibrating massaging unit on the breast pump cup in contact with the breast, it is possible to achieve a direct massage to the breast. A vibrating motor is embedded in the cup body of the breast pump cup, so as to make the structure compact, and make the massage experience comfortable, so that the massage intensity or massage frequency can be conveniently controlled just by controlling current.

### FIELD OF THE INVENTION

The present invention belongs to the technical field of maternal and infant products, in particular to a wearable breast pump having a function of vibration and massage.

### BACKGROUND OF THE INVENTION

A breast pump is a tool having a function of assisting postpartum mothers in sucking breast milk out, which can make it rapid to suction and store the breast milk. With the aid of the breast pump, when a mother has no time while working or going out, she can let her baby get breastfed in time. However, existing breast pumps have a relatively simple function, and in the process of sucking breast milk out, it possibly happens to mothers having different body constitutions that slight pain occurs to their breasts, influencing their feelings of use, and it is possible to affect secretion of breast milk in severe cases. Therefore, one has proposed a breast pump having an additional function of vibration and massage. For example, the Chinese patent (Application number: CN212854209) discloses a breast pump having a function of vibration and massage, which is a kind of breast pumps that need holding by hand, but it is inconvenient to use such a kind of breast pumps due to necessity to control a vibration intensity during use only by way of connecting a hose and an electric conduction wire. In addition, due to modes of wiring and contact, hooking arms and other objects probably happens during use, and loose contact occurs, resulting in intermittent feelings to vibration and massage and influencing use experience. The Chinese patent (Application number: CN111658859A) discloses a wearable breast pump having a function of vibration and massage, including a main unit and a built-in vibrating piece disposed inside the main unit, wherein only after spreading a vibration sensation from the main unit to a breast cup, it is possible to vibrate and massage the breast. The main unit and the breast cup are movably and softly connected to each other in the breast pump, resulting in a distinct reduction in the vibration spreading from the inside of the main unit to the breast cup, and failing to produce an effect of vibration and massage.

Therefore, a wearable breast pump producing an effect of directly vibrating and massaging a breast, having a simple structure and making it easy to use, is required for this industry.

### SUMMARY OF THE INVENTION

The main objective of the present invention is to provide a wearable breast pump having a function of vibration and massage, which belongs to a kind of integrated breast pumps easy to use and carry about, and which has a simple structure and makes it easy to use by way of using a vibrating element mounted on a breast pump cup to massage the breast directly, so as to serve a function in vibration and massage, which can be performed before and after suctioning breast milk or during pumping breast milk, and can effectively relieve pain and discomfort occurring to the breast during pumping, and which can further stimulate and unclog mammary glands and redound to secretion of breast milk.

The wearable breast pump having a function of vibration and massage claimed in the present invention comprises a breast pump cup, a massage portion and a vibrating massaging unit, wherein the massage portion is disposed on the breast pump cup, the vibrating massaging unit is correspondingly disposed on the massage portion, and the vibrating massaging unit produces a vibration under the action of electric current.

In some embodiments, a cup body of the breast pump cup is flexible, and the vibrating massaging unit is embedded in the cup body of the breast pump cup.

In some embodiments, the massage portion is configured to be a plurality of protrusions evenly distributed on the breast pump cup.

In some embodiments, a touching pellet is disposed on each of the protrusions.

In some embodiments, an auxiliary massage protruding portion is arranged between each two of the protrusions circumferentially adjacent to one another.

In some embodiments, a first electricity supply port for connecting an external controller is disposed on the breast pump cup, and the first electricity supply port is electrically connected to the vibrating massaging unit.

In some embodiments, the wearable breast pump further includes a main unit that can be detached therefrom, the main unit has a controller, and a second electricity supply port that is electrically connected to the controller is disposed on the main unit, the main unit is mounted on the wearable breast pump, the first electricity supply port is electrically connected to the second electricity supply port.

In some embodiments, a control key is disposed on the main unit, and configured to control operation modes of the vibration massaging unit, and the main unit further has a display interface, which is configured to display a setting of suctioning breast milk and a setting of vibration.

In some embodiments, the main unit controls a vibration intensity and frequency by means of PWM.

In some embodiments, the first electricity supply port is connected with the second electricity supply port by means of a magnetic waterproof seat.

Compared with the prior art, the wearable breast pump has the following beneficial effect.

In the present invention, by way of arranging the vibrating massaging unit on the breast pump cup in contact with the breast, it is possible to achieve a direct massage to the breast, ensure an appropriate strength of the massage, and avoid the vibration through soft connection from getting weakened. A vibrating motor is embedded in the cup body of the breast pump cup, so as to make the structure compact, and make the massage experience comfortable, so that the massage intensity or massage frequency can be conveniently controlled just by controlling current. Benefiting from that the electricity supply ports contactable with each other are used to provide power for the vibrating massaging unit, it is possible to make the structure simple, and make it easy to assemble and disassemble, and it easy to use.

### BRIEF DESCRIPTION OF THE DRAWINGS

In order to show the details of the present invention and make it easy to understand the principle thereof, it is unnecessary to draw a figure to scale. In different figures, the same symbol represents similar elements. The symbols with different letter suffixes can indicate different examples of the similar elements. The figures indicate the embodiments discussed herein through examples rather than by means of limitations.

FIG. 1 is a 3D diagram of the breast pump.

FIG. 2 is a front view of the breast pump cup of the breast pump.

FIG. 3 is a diagram showing how to arrange the vibrating motor after removing the breast pump cup from the breast pump in FIG. 1.

FIG. 4 is a 3D diagram of the breast pump at another angle of view.

FIG. 5 is a diagram of the breast pump in FIG. 4 from which the main unit is removed.

FIG. 6 is a diagram of the main unit of the breast pump.

FIG. 7 is a module diagram showing how to control vibration.

FIG. 8 is a diagram showing the vibration frequency and intensity of the vibrating piece controlled by PWM.

wherein, 1—breast pump cup; 101—protrusion; 102—auxiliary massage protruding portion; 103—first electricity supply port; 2—breast milk suctioning hole; 3—vibrating motor; 4—electric conduction wire; 5—main unit; 501—second electricity supply port; 502—control key.

### DETAILED DESCRIPTION OF SOME EMBODIMENTS

We shall further describe the technical solution of the present invention as follows in combination with the figures and the examples. There is no limitation of these examples on the present invention. A person skilled in the art should understand that all directional words (for example, above, below, top, bottom, left, right, vertical, horizontal, etc.) are only destined to describe these examples, aid in readers understanding, and do not pose any limitation on the protection scope.

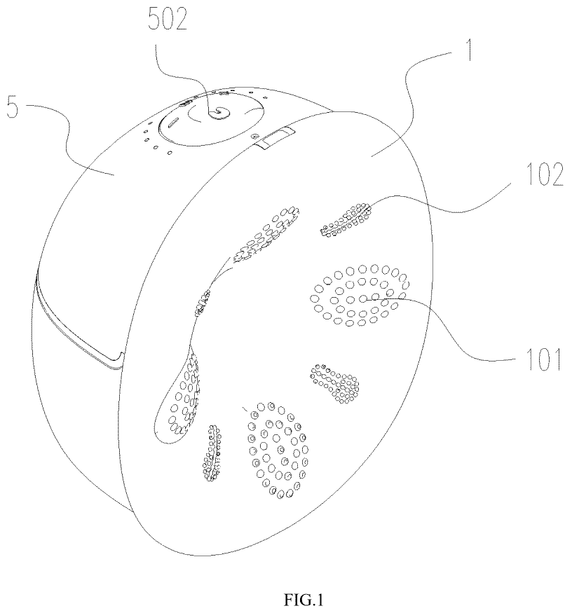

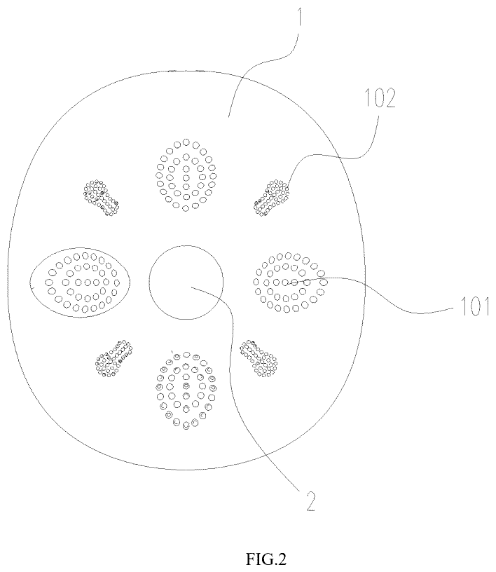

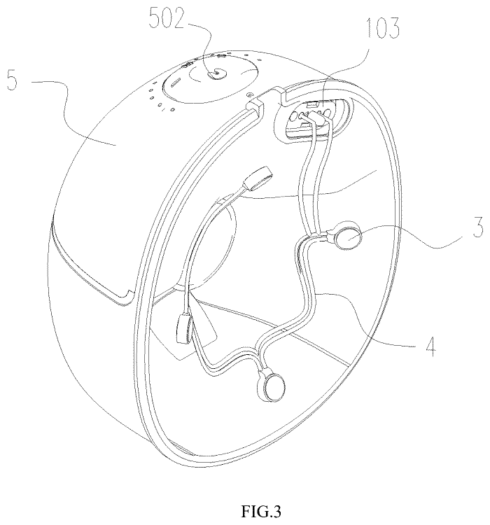

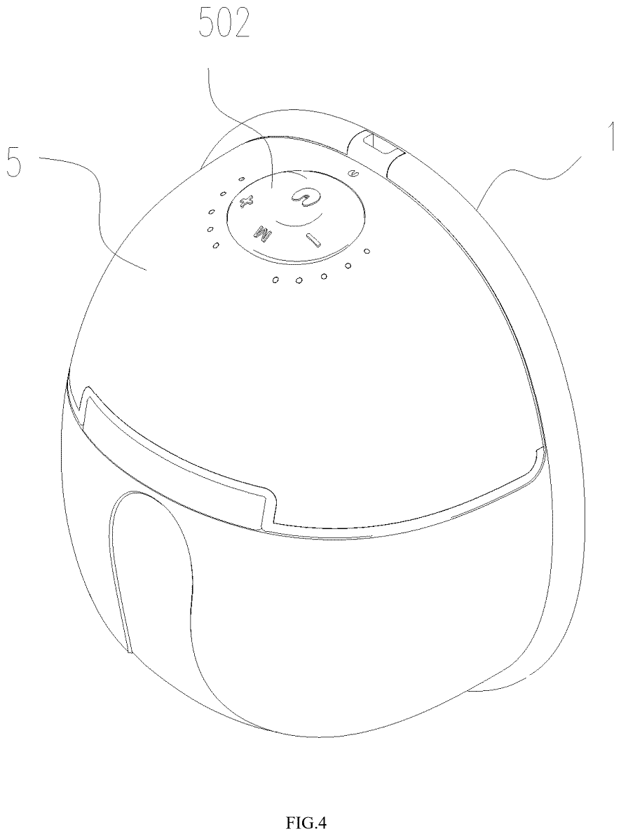

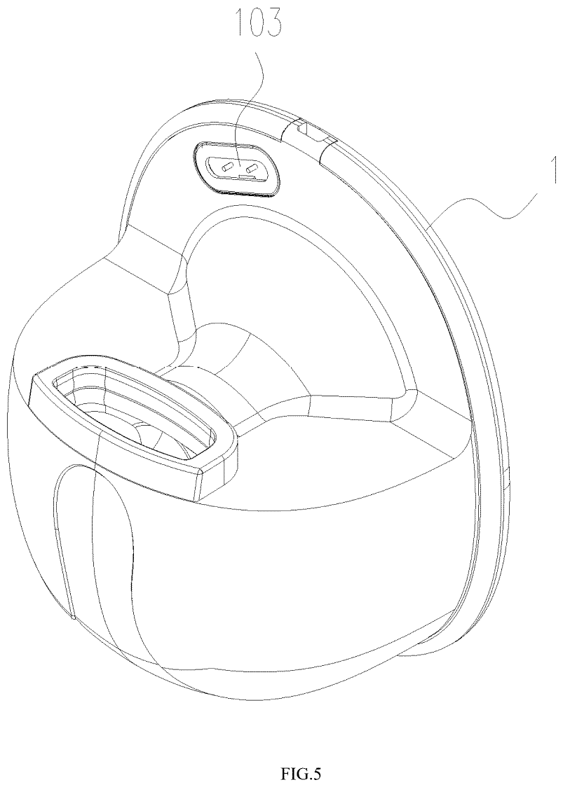

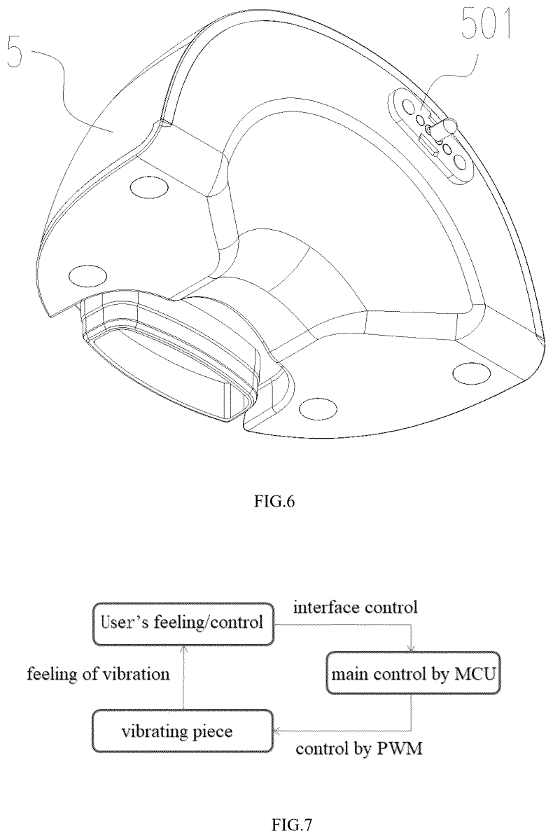

As shown in FIGS. 1-6, the wearable breast pump having a function of vibration and massage claimed in the present invention includes a breast pump cup 1 configured to cover a breast, a breast milk suctioning hole 2 is disposed in the middle part of the breast pump cup 1, so that a nipple is put at the breast milk suctioning hole 2 during use, and under the action of negative pressure the breast milk suctioning hole 2 achieves pumping milk from the breast. A massage portion in contact with the breast is disposed on the breast pump cup 1, and the massage portion is preferred to lie at the position sensitive to stimulation corresponding to the breast or on some acupoints favorable to stimulation, so as to improve the massage effect. A vibrating massaging unit is correspondingly disposed on the massage portion. The vibrating massaging unit in this embodiment is a miniature vibrating motor 3, which produces a vibration under the action of electric current, so as to correspondingly vibrate the massage portion and achieve massaging the breast. The cup body of the breast pump cup 1 is made of flexible material, such as silicone or plastic material, the vibrating motor 3 is embedded in the cup body of the breast pump cup 1, so as to make the structure compact and simple, make the massage experience comfortable, and male the use convenient, so that the massage intensity or massage frequency can be conveniently controlled just by controlling current. As a replacement, a vibrating piece such as a piezoelectric vibrating sheet may also serve as the vibrating massaging unit.

Further, the massage portion is configured to be four protrusions 101 arranged on the breast pump cup 1, and these protrusions 101 are circumferentially and evenly distributed around the breast milk suctioning hole 2. The vibrating motor 3 is embedded inside the protrusion 101. A plurality of touching pellets are arranged on the protrusions 101 to improve the stimulating effect of massage.

Further, an auxiliary massage protruding portion 102 having a small size is arranged between each two of the protrusions 101 circumferentially adjacent to one another, so that when the breast pump cup 1 vibrates, the auxiliary massage protruding portion 102 also play the role of auxiliary massage.

Further, a first electricity supply port 103 for connecting an external controller is disposed on the breast pump cup 1, and the first electricity supply port 103 is electrically connected to the vibrating motor 3 through an electric conduction wire 4, so as to provide operation current to the vibrating motor 3 through the first electricity supply port 103.

Further, the wearable breast pump further includes a main unit 5 that can be detached therefrom, and a rechargeable battery serving as a power source, a controller for controlling electrical devices, and a vacuum pump for making vacuum from the breast milk suctioning hole 2 are integrated inside the main unit. A second electricity supply port 501 that is electrically connected to the controller is further disposed on the main unit 5, the main unit 5 is mounted on the wearable breast pump, the first electricity supply port 103 is electrically connected to the second electricity supply port 501, so as to supply electricity to the vibrating motor 3. The controller controls operation of the vibrating motor 3 by way of regulating its current values, for example, the controller can control its vibration intensity and vibration frequency. A control key 502 is disposed on the main unit 5, so as to control operation modes of the vibrating motor 5 by way of a woman manipulating the control key 502 with her fingers. An elastic contact point is correspondingly disposed on the first electricity supply port and the second electricity supply port respectively, so as to ensure electrical contact. The first electricity supply port is connected with the second electricity supply port by means of a waterproof seat, that is, the first electricity supply port and the second electricity supply port each have a magnetic pole opposite to each other, so that the two ports are tightly combined together under the action of magnetic force. It is possible for the breast pump to improve the connection tightness of the contacts, have waterproof performance and make it easy to disassemble and assemble, by means of magnetic absorption.

Further, on the base of the display interface on the main unit 5, it is possible to know a current vibration state by means of an light indicating a setting of vibration, and switch and adjust the vibration intensity and the vibration frequency by means of a button for controlling vibration.

The controller of the main 5 in this embodiment is also used for controlling operation of the vacuum pump.

As an embodiment, in the wearable breast pump having a function of vibration and massage, a means for vibrating is disposed in a horn hood and electrically connected to an electricity supply unit. In this embodiment, preferably the means for vibrating is a vibrating motor.

The following table 1 shows design requirements for the battery capacity required for the vibration of the wearable breast pump having a function of vibration and massage provided in this embodiment in a preset time length of operation.

TABLE 1
DESIGN AND CALCULATION FOR VIBRATION

| | |
|---|---|
| operation time length for the | 90 min |
| breast pump | |
| parameters of the vibrating | Of the motor, the rated voltage is 3 V and the rated current is |
| motor | 80 mA, and if 4 motors are used for vibration and massage, |
| | and the total output is: 3*0.08*4 = 0.96 W ≈ 1 W |
| battery capacity required for | 1 w*1.5 h = 1.5 wh, at least 3.7 V/400 mAh in battery capacity is |
| all motors to work for 90 min | required. |
| total required battery | Because the breast pump needs 3.7 V/1000 mAh for working |
| capacity | for 90 min, the total battery capacity is to be at least |
| | 3.7 V/1400 mAh. |

Note:
the above calculations only give theoretical values, and there will be some deviations in practice.

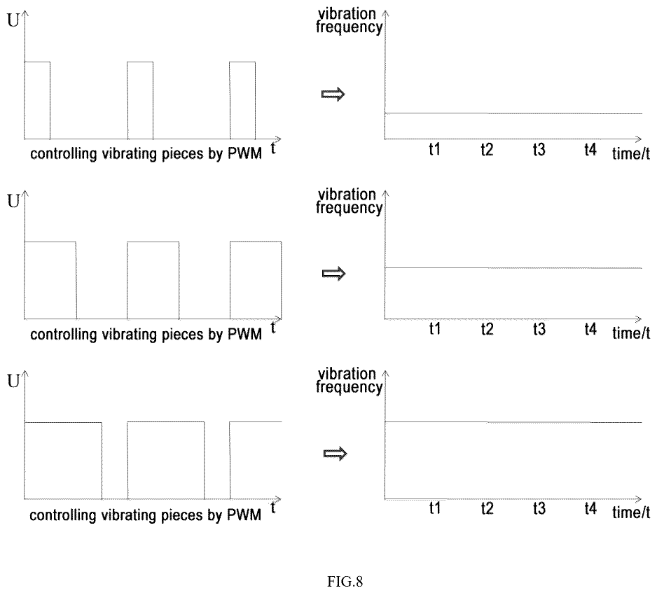

FIG. 7 is a module diagram showing how to control vibration. At the position of the control key a display interface, such as a LED display, a LCD display and a LED indicator light, may be correspondingly disposed.

FIG. 8 is a diagram showing the vibration frequency and intensity of the vibrating piece controlled by PWM. The controller can actualize its control though PWM of a MCU, which can achieve control to different vibration frequencies and vibration feelings, allowing users to choose different settings of vibration according to their own User's feelings.

The above is only a specific embodiment of the present invention. It should be noted that any modifications, equivalent substitutions and changes made within the essence and idea of the present invention shall fall within the scope protection of the present invention.

What is claimed is:

1. A wearable breast pump having a function of vibration and massage, comprising a breast pump cup, wherein a massage portion is disposed on the breast pump cup, a vibrating massaging unit is correspondingly disposed on the massage portion, and the vibrating massaging unit produces a vibration under the action of electric current.

2. The wearable breast pump having a function of vibration and massage according to claim 1, wherein a cup body of the breast pump cup is flexible, and the vibrating massaging unit is embedded in the cup body of the breast pump cup.

3. The wearable breast pump having a function of vibration and massage according to claim 1, wherein the massage portion is configured to be a plurality of protrusions evenly distributed on the breast pump cup.

4. The wearable breast pump having a function of vibration and massage according to claim 3, wherein a touching pellet is disposed on each of the protrusions.

5. The wearable breast pump having a function of vibration and massage according to claim 3, wherein an auxiliary massage protruding portion is arranged between each two of the protrusions circumferentially adjacent to one another.

6. The wearable breast pump having a function of vibration and massage according to claim 1, wherein a first electricity supply port for connecting an external controller is disposed on the breast pump cup, and the first electricity supply port is electrically connected to the vibrating massaging unit.

7. The wearable breast pump having a function of vibration and massage according to claim 6, wherein the wearable breast pump further comprises a main unit that can be detached therefrom, the main unit has a controller, and a second electricity supply port that is electrically connected to the controller is disposed on the main unit; when the main unit is mounted on the wearable breast pump, the first electricity supply port is electrically connected to the second electricity supply port.

8. The wearable breast pump having a function of vibration and massage according to claim 7, wherein a control key is disposed on the main unit, and configured to control operation modes of the vibration massaging unit, and the main unit further has a display interface, which is configured to display a setting of suctioning breast milk and a setting of vibration.

9. The wearable breast pump having a function of vibration and massage according to claim 7, wherein the main unit controls a vibration intensity and frequency by means of PWM.

10. The wearable breast pump having a function of vibration and massage according to claim 7, wherein the first electricity supply port is connected with the second electricity supply port by means of a magnetic waterproof seat.
